(19) United States
(12) Patent Application Publication
(10) Pub. No.: US 2006/0210518 A1
(43) Pub. Date: Sep. 21, 2006

(54) Composition containing a urea compound and an oxyalkylenated compound

(75) Inventor: Odile Aubrun-Sonneville, Antony (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/365,524

(22) Filed: Mar. 2, 2006

Related U.S. Application Data
(60) Provisional application No. 60/665,392, filed on Mar. 28, 2005.

(30) Foreign Application Priority Data
Mar. 17, 2005 (FR) 05 50685

(51) Int. Cl. A61K 8/73; A61K 8/40

(52) U.S. Cl. 424/70.13; 424/70.7; 424/70.21; 424/73

(57) ABSTRACT

The invention relates to a cleansing composition containing, in an aqueous medium, (1) at least one nonionic oxyalkylenated compound, (2) at least one compound of formula (I) below: 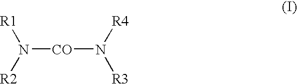 in which: R1, R2, R3 and R4 represent, independently of each another, a hydrogen atom, a C1-C4 alkyl group or a C2-C6 hydroxyalkyl group possibly containing from 1 to 5 hydroxyl groups, in which at least one of the radicals R1 to R4 represents a hydroxyalkyl group, and also the salts, solvates and isomers thereof, and (3) at least one foaming surfactant. Use of the composition as a skin cleansing product, etc.

### REFERENCE TO PRIOR APPLICATIONS

This application claims priority to U.S. provisional application 60/665,392 filed Mar. 28, 2005, and to French patent application 0550685 filed Mar. 17, 2005, both incorporated herein by reference.

### FIELD OF THE INVENTION

The invention relates to a composition containing, in an aqueous medium, a hydroxy urea compound, a nonionic oxyalkylenated compound and a foaming surfactant, and to its uses for cleansing and/or removing makeup from human skin, the lips and/or the hair.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Cleansing of the skin is very important for facial care, and it should be as efficient as possible since greasy residues such as excess sebum, residues of cosmetic products used daily and makeup products accumulate in the folds of the skin and can block the skin pores and lead to the formation of spots.

It is known practice to use foaming gels to cleanse the skin. These products are aqueous solutions of foaming surfactants, which are used for their detergent properties, and are generally thickened with polymers, in order to facilitate their handling. Nonionic oxyalkylenated derivatives are commonly used for thickening foaming gels since they lead to transparent products whose foam quality remains good.

However, these products often have the drawback of being lacking in comfort when applied by giving a sensation of tautness and dryness. In point of fact, one of the major difficulties in the formulation of foaming products consists in developing products that have very good foaming power to ensure good cleansing, and which can be rinsed off easily, while at the same time respecting the skin barrier, i.e. without drying out the skin.

One means for improving the sensation of comfort and moisturization is to add moisturizers such as glycerol. However, this type of derivative has a tendency to fluidize the foaming compositions containing it, which leads to a modification of the texture and may impair the qualities of the compositions, which become more difficult to handle.

There is thus still a need for cosmetic cleansing compositions that do not dry out the skin but nevertheless have a texture that is suited to the intended use and especially a satisfactory viscosity.

### SUMMARY OF THE INVENTION

The inventor has discovered, surprisingly, that it is possible to obtain cleansing products, which provide a moisturizing effect, without compromising the texture of the composition, by introducing at least one urea derivative into foaming gels thickened with a nonionic oxyethylenated compound. The foaming gel contains one or more foaming surfactants.

### DETAILED DESCRIPTION OF THE PREFERRED EMBODIMENTS

One subject of the invention is a composition comprising, in an aqueous medium, (1) at least one nonionic oxyalkylenated compound, (2) at least one compound of formula (I) below: 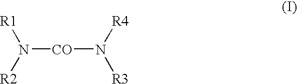
in which:
R1, R2, R3 and R4 represent, independently of each another, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts, solvates and isomers thereof, and (3) at least one foaming surfactant.

The composition of the invention has the advantage of not drying out the skin while at the same time having satisfactory viscosity. Without being bound by a particular theory, it is believed that the use of the compound of formula (I) as a moisturizer makes it possible, unexpectedly, to maintain the thickening power of oxyethylenated compounds, which is not possible with other moisturizers such as glycerol. No document suggests that the compounds of formula (I) make it possible to maintain the gelling power of nonionic oxyethylenated compounds in foaming products.

In this patent application, the term “aqueous medium” means a medium containing water and optionally one or more water-soluble organic solvents. In this aqueous medium, the amount of water is preferably at least 20% by weight; it preferably ranges from 20% to 95% by weight, and better still from 30% to 90% by weight and even better still from 40% to 85% by weight relative to the total weight of the composition.

In addition, since the composition of the invention is preferably a cosmetic composition, it preferably contains a physiologically acceptable medium. The term “physiologically acceptable medium” means herein a medium that is compatible with the skin, mucous membranes and the eyes. It is preferably a cosmetically acceptable medium, i.e. a medium that also has a pleasant colour, odour and feel and that does not cause any unacceptable discomfort (stinging, tautness or redness) liable to put the consumer off using this composition.

The compositions according to the invention may be in any form, including in the form of thickened solutions, gels, milks or more or less thick creams, and they may or may not flow under their own weight according to their viscosity. The viscosity measured at 25° C. using a Rheomat 180 viscometer at 200 rpm (revolutions per minute) may range, for example, from 0.5 to 100 Pa·s (5 to 1000 poises) and more particularly between 0.8 and 50 Pa·s. The Rheomat 180 machine is equipped with a different spindle according to the viscosity, for example a No. 3 spindle for the viscosity range from 0.2 to 4 Pa·s and a No. 4 spindle for the range of viscosities greater than 2 Pa·s. This viscosity is generally measured 10 minutes after starting the rotation of the spindle.

Advantageously, the pH of the aqueous medium is compatible with keratin materials and especially with the skin. This pH preferably ranges from 3 to 8.5, better still from 3.5 to 8 and preferentially from 5 to 7.5.

The cleansing composition of the invention constitutes a foaming composition that is rinsed off after application to the skin.

Hydroxy Urea Compound

The at least one urea compound present in the composition according to the invention is at least one compound of formula (I) below: 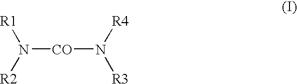
in which:
R1, R2; R3 and R4 represent, independently of each other, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts, solvates and isomers thereof.

For the compounds of formula (I):

preferably, R1 denotes a C2-C6 hydroxyalkyl group and R2, R3 and R4 denote, independently of each other, a hydrogen atom or a C1-C4 alkyl group;

preferentially, R1 denotes a C2-C6 hydroxyalkyl group comprising from 1 to 5 hydroxyl groups and especially 1 hydroxyl group, and R2, R3 and R4 denote a hydrogen atom;

more preferentially, R1 denotes a C2-C4 hydroxyalkyl group comprising one hydroxyl group and R2, R3 and R4 denote a hydrogen atom.

C1-C4 alkyl groups that may especially be mentioned include methyl, ethyl, n-propyl, isopropyl, n-butyl, isobutyl and tert-butyl groups.

Among the C1-C6 hydroxyalkyl groups that are preferred are those containing only one hydroxyl group and in particular hydroxyethyl, hydroxypropyl, hydroxybutyl, hydroxypentyl and hydroxyhexyl groups.

Salts of such compounds that may be mentioned include the salts of mineral acids, such as sulfuric acid, hydrochloric acid, hydrobromic acid, hydriodic acid, phosphoric acid and boric acid. Mention may also be made of the salts of organic acids, which may comprise one or more carboxylic acid, sulfonic acid or phosphonic acid groups. They may be linear, branched or cyclic aliphatic acids or alternatively aromatic acids. These acids may also comprise one or more hetero atoms chosen from O and N, for example in the form of hydroxyl groups. Mention may especially be made of propionic acid, acetic acid, terephthalic acid, citric acid and tartaric acid.

The term “solvate” means a stoichiometric mixture of the said compound of formula (I) with one or more water molecules or organic solvent molecules, such as a mixture derived from the synthesis of the compound of formula (I).

Preferred compounds of formula (I) that may be mentioned include N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxy-hexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)-methyl]urea; N-ethyl-N′-(2-hydroxyethyl)urea; N,N-bis(2-hydroxyethyl)urea; N,N′-bis(2-hydroxyethyl)-urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxy-ethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′N′-tetrakis(2-hydroxyethyl)-urea; N′N′-bis(2-hydroxyethyl)-N′,N′-bis(2-hydroxy-propyl)urea; and mixtures thereof.

Preferably, the compound of formula (I) is N-(2-hydroxyethyl)urea.

The compounds of formula (I) are known compounds and are described especially in patent application DE-A-2 703 185. Among these, N-(2-hydroxyethyl)urea is also commercially available, in the form of a mixture at 50% by weight in water, from the company National Starch under the trade name Hydrovance®.

The compound of formula (I) may be present in the composition according to the invention in any amount (of active material) including, e.g., 0.1% to 50% by weight, preferably ranging from 0.1% to 20% by weight and preferentially ranging from 0.1% to 10% by weight relative to the total weight of the composition.

Oxyalkylenated Compounds

The composition according to the invention comprises one or more nonionic oxyalkylenated compounds, which are compounds comprising at least one group chosen from ethylene oxide (EO) groups, propylene oxide (PO) groups and mixtures thereof (EO PO). The compounds may thus be oxyethylenated compounds, oxypropylenated compounds or oxyethylenated/-oxypropylenated compounds. These compounds are not surfactants, but have thickening properties.

The amount of nonionic oxyalkylenated compound(s) in the composition of the invention, by weight of active material, is not limited and may range, for example, from 0.1% to 15% by weight, better still from 0.2% to 10% by weight and even better still from 0.2% to 5% by weight relative to the total weight of the composition.

The nonionic oxyalkylenated compounds may be chosen especially from polyethylene glycols, fatty acid esters of polyethylene glycol and/or of polypropylene glycol, fatty alkyl ethers of polyethylene glycol and/or of polypropylene glycol, alkyl- or acyl-alkoxylated derivatives and especially of polyol, glyceryl triesters of oxyalkylenated and especially oxyethylenated fatty acids, oxyethylenated or oxypropylenated fatty amide derivatives, and oxyethylenated urethane derivatives modified with alkyl chains, and mixtures thereof.

1. The polyethylene glycols that may be used in the composition of the invention include ethylene oxide polycondensates. Preferably, these polyethylene glycols have a number of ethylene oxide (EO) units of greater than 1000. The ethylene oxide number may range, for example, from 1000 to 50 000 and preferably from 5000 to 10 000. Examples of polyethylene glycols that may be mentioned include the polyethylene glycol comprising 7000 EO (CTFA name: PEG-7M), for instance the product sold under the name Polyox WSR N-750® by the company Amerchol, the polyethylene glycol comprising 14 000 EO (CTFA name: PEG-14M), for instance the product sold under the name Polyox WSR 205 by the company Amerchol, and the polyethylene glycol comprising 20 000 EO (CTFA name: PEG-20M), for instance the product sold under the name Polyox WSR 1105® by the company Amerchol.

2. The fatty acid esters of polyethylene glycol and/or of polypropylene glycol include condensates of polyethylene glycol and/or polypropylene glycol with one or more fatty acids. These are compounds of formula (II):
RCOO—(OE)m-(OP)n═R′ (II)
in which 0<m<300 and 0<n<300 and m+n>6, R and R′ represent, independently of each other, hydrogen or a saturated or unsaturated, linear or branched, hydroxylated or non-hydroxylated alkyl chain containing from 1 to 30 carbon atoms and preferably from 12 to 22 carbon atoms, or an aryl chain, on condition that R and R′ are not simultaneously hydrogen.

As fatty acid esters of polyethylene glycol and/or of polypropylene glycol, examples that may be mentioned include polyethylene glycol (150 EO) distearate such as the product sold under the name Atlas G-1821® by the company Uniqema, PEG-150 dibehenate such as the product sold under the name Ethox PEG 6000 dibehenate® by the company Ethox, polyethylene glycol (120 EO) palmito stearate, such as the product sold under the name Stearate 6000 WL 1644® by the company Gattefosse, the copolymer of polyethylene glycol (30 EO) and of 12-hydroxystearate acid, such as the product sold under the name Arlacel P135® by the company Uniqema, and polyethylene glycol (40 EO) stearate such as the product sold under the name Myrj 52® by the company Uniqema.

In the case where, in formula (II),

R═R′═H, an example that may be mentioned is the polyoxyethylene/polyoxypropylene (17 EO/6 PO) random copolymer sold under the reference Ucon 75-H-450® by the company Amerchol. Molecules comprising more EO and/or more PO are not excluded.

3. The fatty alkyl ethers of polyethylene glycol and/or of polypropylene glycol include condensates of polyethylene glycol and/or polypropylene glycol with one or more fatty alcohols. They include compounds of formula (III):
R—(OE)m-(OP)n-R′ (III)
in which 0<m<300 and 0<n<300 and m+n>6, R and R′ represent, independently of each other, hydrogen or a saturated or unsaturated, linear or branched, hydroxylated or non-hydroxylated alkyl chain containing from 1 to 30 carbon atoms and preferably from 12 to 22 carbon atoms, or an aryl chain, on condition that R and R′ are not simultaneously hydrogen.

Examples of fatty alkyl ethers of polyethylene glycol that may especially be mentioned include oxyethylenated (4 EO) lauryl alcohol, such as the product sold under the name Brij 30® by the company Uniqema, oxyethylenated (30 EO) cetyl alcohol such as the product sold under the name Nikkol BC-30TX® by the company-Nikkol, oxyethylenated (15 EO) oleyl alcohol such as the product sold under the name Nikkol BO-15TX® by the company Nikkol, oxyethylenated (50 EO) oleyl alcohol such as the product sold under the name Nikkol BO-50® by the company Nikkol, oxyethylenated (10 EO) behenyl alcohol such as the product sold under the name Mergital B 10® by the company Nikkol, oxyethylenated (30 EO) behenyl alcohol such as the product sold under the name Nikkol BB-30® by the company Nikkol, oxyethylenated (12 EO) lauryl alcohol such as the product sold under the name Rewopal 12® by the company Goldschmidt, oxyethylenated (23 EO) lauryl alcohol such as the product sold under the name Simulsol P 23® by the company SEPPIC, oxyethylenated (20 EO) 2-octyldodecyl alcohol such as the product sold under the name Octyldodeceth-20® by the company Stearinerie Dubois, oxyethylenated (20 EO) isocetyl alcohol such as the product sold under the name Arlasolve 200 US® by the company Uniqema, oxyethylenated (10 EO) oleyl alcohol such as the product sold under the name Brij 97® by the company Uniqema, oxyethylenated (20 EO) oleyl alcohol such as the product sold under the name Brij 98® by the company Uniqema, oxyethylenated (100 EO) stearyl alcohol such as the product sold under the name Brij 700® by the company Uniqema, and oxyethylenated (21 EO) stearyl alcohol such as the product sold under the name Brij 721® by the company Uniqema.

An example of a polypropylene glycol ether that may especially be mentioned is oxypropylenated (3 PO) myristyl alcohol such as the product sold under the name Promyristyl PM-3® by the company Croda.

Examples of polyethylene glycol/polypropylene glycol ethers that may especially be mentioned include oxyethylenated (5 EO) oxypropylenated (5 PO) lauryl alcohol such as the product sold under the name Aethoxal B® by the company Cognis, oxyethylenated (20 EO) oxypropylenated (5 PO) cetyl alcohol such as the product sold under the name Procetyl AWS® by the company Croda, oxyethylenated (26 EO) oxypropylenated (26 PO) butyl alcohol such as the product sold under the name PPG-26-Buteth-26® by the company Goldschmidt, oxyethylenated (26 EO) oxypropylenated (26 PO) butyl alcohol such as the product sold under the name Varonic APEB® by the company Goldschmidt, oxyethylenated (30 EO) oxypropylenated (6 PO) decyltetradecanol such as the product sold under the name Nikkol PEN-4630® by the company Nikkol, and oxyethylenated (25 EO) oxypropylenated (25 PO) lauryl alcohol such as the product sold under the name ADF-Oleile® by the company Vevy.

4. The alkoxylated alkyl or acyl derivatives of polyol may especially be ethoxylated alkyl or acyl derivatives of polyol, for example oxyethylenated derivatives of fatty acid esters of polyol or oxyethylenated derivatives of fatty alkyl ethers of polyol, and especially oxyethylenated derivatives of fatty acid esters or of fatty alkyl ethers and of glycerol, sorbitol, glucose or pentaerythritol.

Examples of derivatives of this type that may be mentioned include oxyethylenated (78 EO) glyceryl cocoate such as the product sold under the name Simulsol CG by the company SEPPIC, oxyethylenated (120 EO) methylglucose dioleate (CTFA name: PEG-120 methylglucose dioleate) such as the product sold under the name Glucamate DOE-120 Vegetal® by the company Amerchol, oxyethylenated (40 EO) sorbitan heptaoleate such as the product sold under the name Arlatone T® by the company Uniqema, oxyethylenated (10 EO) polyglyceryl (2 mol of glycerol) laurate such as the product sold under the name HOE S 3495® by the company Clariant, oxyethylenated (60 EO) glyceryl isostearate such as the product sold under the name Emalex GWIS-160® by the company SACI-CFPA, oxyethylenated (20 EO) glyceryl monostearate such as the product sold under the name Cutina E 24® by the company Cognis, oxyethylenated (200 EO) glyceryl stearate such as the product sold under the name Simulsol 220 TM® by the company SEPPIC, oxyethylenated (150 EO) pentaerythrityl tetrastearate such as the product sold under the name Crothix® by the company Croda, and oxyethylenated (160 EO) sorbitan tristearate such as the product sold under the name Rheodol TW IS399C by the company Kao Chemicals.

5. Examples of oxyalkylenated glyceryl triesters of fatty acids that may be mentioned include oxyethylenated (6 EO) caprylic/capric acid glycerides, such as the product sold under the name Softigen 767® by the company Condea, and oxyethylenated (50 EO) olive oil such as the product sold under the name Crovol O-70 by the company Croda.

6. Oxyalkylenated fatty acid amide derivatives that may especially be mentioned include oxypropylenated fatty acid amides, for instance PPG-2 hydroxyethyl cocamide and mixtures containing it, such as the products sold by Uniqema under the name Promidium, especially Promidium CO.

7. Examples of oxyethylenated urethane derivatives modified with alkyl chains that may be mentioned include those of formulae (III) and (IV):
R₁NH—CO—(OCH₂CH₂)ₐ—[O—CO—NR₄—R₃—CO—(OCH₂CH₂)ₐ]_{b}—O—CO—NHR₂ (III)
R₅—(OCH₂CH₂)ₙ—O—CO—NH—R₆—NH—CO—(OCH₂CH₂)ₙ—OR₅ (IV)
in which the radicals R₁, R₂ and R₅ represent a C₁₋₁₈ alkyl group; R₃ and R₆ represent a linear, cyclic or aromatic C₄₋₃₆ hydrocarbon-based radical; R₄ represents a hydrogen atom or a C₁₋₆ alkyl radical, preferably a hydrogen atom; a and n are integers ranging from 90 to 600, and b is an integer ranging from 1 to 4.

These are, for example, water-soluble polymers obtained by addition reaction of diisocyanates (HMDI: hexamethylene diisocyanate) with diols (polyethers or polyesters) and ending with hydrophobic groups derived from ethoxylated or ethoxylated/-propoxylated fatty alcohols. This is the case, for example, for Nuvis FX 1100 sold by the company Elementis, which is an oxyethylenated (100 EO) stearyl alcohol/polyethylene glycol (136 EO)/hexamethylene diisocyanate copolymer (CTFA name: steareth-100/-PEG-136/HMDI copolymer).

According to one preferred embodiment of the invention, the nonionic oxyalkylenated compound is chosen from polyethylene glycols, oxyethylenated derivatives of fatty acid esters or ethers of polyol, especially with or without methyl glucose or pentaerythritol, oxyethylenated or oxypropylenated fatty acid amide derivatives, and oxyethylenated urethane derivatives modified with alkyl chains, and mixtures thereof.

Foaming Surfactant

The foaming composition according to the invention contains at least one foaming surfactant that gives the composition its foaming nature. This surfactant may be chosen from nonionic, anionic, amphoteric and zwitterionic foaming surfactants, and mixtures thereof.

Foaming surfactants are detergents and differ from emulsifiers by their HLB (hydrophilic lipophilic balance), the HLB being the ratio between the hydrophilic part and the lipophilic part in the molecule. The term HLB is well known to those skilled in the art and is described, for example, in “The HLB system. A time-saving guide to Emulsifier Selection” (published by ICI Americas Inc.; 1984). For emulsifiers, the HLB generally ranges from 3 to 8 for the preparation of W/O emulsions and from 8 to 18 for the preparation of O/W emulsions, whereas foaming surfactants generally have an HLB of greater than 20.

The amount of foaming surfactant is not limited and may range, for example, by weight of active material, from 1% to 50% by weight and better still from 2% to 20% by weight relative to the total weight of the composition.

1. Nonionic surfactants:

The nonionic surfactants may be chosen, for example, from alkyl polyglucosides (APG), maltose esters, polyglycerolated fatty alcohols, and glucamine derivatives, for instance 2-ethylhexyloxycarbonyl-N-methylglucamine, and mixtures thereof.

Alkylpolyglucosides that are preferably used include those containing an alkyl group containing from 6 to 30 carbon atoms and preferably from 8 to 16 carbon atoms, and containing a hydrophilic group (glucoside) preferably comprising 1.2 ro 3 glucoside units. Examples of alkylpolyglucosides that may be mentioned include decylglucoside (Alkyl-C9/C11-polyglucoside (1.4)) for instance the product sold under the name Mydol 10® by the company Kao Chemicals, the product sold under the name Plantaren 2000 UP® by the company Cognis, and the product sold under the name Oramix NS 10® by the company SEPPIC; caprylyl/capryl glucoside, for instance the product sold under the name Oramix CG 110® by the company SEPPIC; lauryl glucoside, for instance the products sold under the names Plantaren 1200 N® and Plantacare 1200® by the company Cognis; cocoglucoside, for instance the product sold under the name Plantacare 818/UP® by the company Cognis; and mixtures thereof.

The maltose derivatives are, for example, those described in document EP-A-566 438, such as O-octanoyl-6′-D-maltose or O-dodecanoyl-6′-D-maltose described in document FR-2 739 556.

Among the polyglycerolated fatty alcohols that may be mentioned is polyglycerolated dodecanediol (3.5 mol of glycerol), this product being sold under the name Chimexane NF® by the company Chimex.

2. The anionic surfactants may be chosen, for example, from soaps (alkali metal salts of fatty acids), carboxylates, acylamino acids, amidoether carboxylates, alkyl polyaminocarboxylates, alkyl ether sulfates such as sodium laureth sulfates, alkyl sulfonates, isethionates, alkyl methyltaurate, alkyl sulfosuccinates, alkyl sulfoacetates and alkyl phosphates (monoalkyl or dialkylphosphates), salts thereof, and mixtures thereof.

Carboxylates that may especially be mentioned include alkyl glycol carboxylic acids (or 2-(2-hydroxyalkyloxyacetic acids)) and salts thereof, for instance sodium lauryl glycol carboxylate, sold under the names Beaulight Shaa® or Beaulight LCA-25N® by the company Sanyo (CTFA name: sodium lauryl glycol carboxylate), or its corresponding acid form sold under the name Beaulight Shaa (acid form)® by the company Sanyo.

An example of an acylamino acid that may be mentioned is sodium cocoylglycinate sold by the company Ajinomoto under the name Amilite GCS 12.

An example of an alkyl phosphate that may be mentioned is lauryl phosphate, sold by the company Kao under the name MAP 20.

3. The amphoteric and zwitterionic foaming surfactants may be chosen, for example, from betaine derivatives including amidopropylbetaines, amphoacetates and amphodiacetates, and hydroxylsultaines, and mixtures thereof.

Examples of betaine derivatives that may be mentioned include cocobetaine, for instance the product sold under the name Dehyton AB-30® by the company Cognis; laurylbetaine, for instance the product sold under the name Genagen KB® by the company Clariant; oxyethylenated (10 EO) laurylbetaine, for instance the product sold under the name Lauryl ether (10 OE) betaine® by the company Shin Nihon Rica; oxyethylenated (10 EO) stearylbetaine, for instance the product sold under the name Stearyl ether (10 OE) betaine® by the company Shin Nihon Rica; the cocamidopropyl betaine sold, for example, under the name Velvetex BK 35® by the company Cognis; the undecylenamidopropyl betaine sold, for example, under the name Amphoram U® by the company Ceca; and mixtures thereof.

Examples of amphoacetates and amphodiacetates that may be mentioned include N-disodium N-cocoyl-N-carboxymethoxyethyl-N-carboxymethylethylenediamine (CTFA name: disodium cocamphodiacetate), for instance the product sold under the name Miranol C2M Concentrate NP® by the company Rhodia Chimie; N-sodium N-cocoyl-N-hydroxyethyl-N-carboxymethylethylenediamine (CTFA name: sodium cocamphoacetate), and mixtures thereof.

At least one of the foaming surfactants is preferably chosen from alkylpolyglucosides, betaine derivatives, alkyl glycol carboxylic acids and salts thereof, alkyl ether sulfates, alkyl phosphates, amphodiacetates, amphoacetates and alkylglycinates, and mixtures thereof.

Adjuvants

As indicated above, the aqueous medium of the composition according to the invention may contain, besides water, one or more water-soluble solvents chosen from lower alcohols (monoalcohols) containing from 1 to 6 carbon atoms, such as ethanol; and polyols such as glycerol, glycols, for instance butylene glycol, isoprene glycol, propylene glycol, polyethylene glycols such as PEG-8; sorbitol; sugars such as glucose, fructose, maltose, lactose and sucrose; and mixtures thereof, provided that these compounds do not impair the desired properties of the composition according to the invention. The amount of solvent(s) in the composition of the invention is not particularly limited and may range, for example, from 0.1% to 30% by weight, preferably from 0.5% to 20% by weight, better still from 0.5% to 15% by weight and even better still from 0.5% to 10% by weight relative to the total weight of the composition.

The compositions of the invention may also contain adjuvants chosen from those usually used in cosmetics. Examples of adjuvants that may be mentioned include fragrances, preserving agents, sequestrants (EDTA), pigments, fillers and especially exfoliant fillers, soluble dyes, sunscreens, cosmetic or dermatological active agents such as moisturizers, for instance hyaluronic acid; ceramides; water-soluble or liposoluble vitamins, for instance vitamin C and derivatives thereof such as vitamin CG; antiseptics; antiseborrhoeic agents; antimicrobial agents such as benzoyl peroxide, salicylic acid, triclosan, azeleic acid; optical brighteners. The amounts of these various adjuvants are are not limited and include those conventionally used in the field under consideration, for example from 0.01% to 5% of the total weight of the composition. These adjuvants and the concentrations thereof preferably are such that they do not modify the property desired for the composition of the invention.

Examples of exfoliants that may be mentioned include exfoliant or scrubbing particles of mineral, plant or organic origin. Thus, it is possible to use, for example, polyethylene beads or powder, Nylon powder, polyvinyl chloride powder, pumice, ground apricot kernel or walnut husk, sawdust, glass beads and alumina, and mixtures thereof. These particles may be present in any amount, ranging, for example, from 0.5% to 30% by weight, preferably from 1% to 20% by weight and better still from 1% to 10% by weight relative to the total weight of the composition. When the composition contains exfoliant particles, it may especially constitute a composition for scrubbing facial or body skin.

The compositions of the invention may also contain adjuvants usually used in the field of foaming cleansing agents, for instance polymers. Although these polymers may be nonionic, they are preferably chosen from anionic polymers and cationic polymers, and mixtures thereof.

Cationic or amphoteric polymers that may especially be mentioned include those of the Polyquaternium type (CTFA name), which give the foaming cream softness and creaminess. These polymers may preferably be chosen from the following polymers:
Polyquaternium 5, such as the product Merquat 5 sold by the company Calgon;
Polyquaternium 6, such as the product Salcare SC 30 sold by the company Ciba, and the product Merquat 100 sold by the company Calgon;
Polyquaternium 7, such as the products Merquat S, Merquat 2200 and Merquat 550 sold by the company Calgon; and the product Salcare SC 10 sold by the company Ciba;
Polyquaternium 10, such as the product Polymer JR400 sold by the company Amerchol;
Polyquaternium 11, such as the products Gafquat 755, Gafquat 755N and Gafquat 734 sold by the company ISP;
Polyquaternium 15, such as the product Rohagit KF 720 F sold by the company Rohm;
Polyquaternium 16, such as the products Luviquat FC905, Luviquat FC370, Luviquat HM552 and Luviquat FC550 sold by the company BASF;
Polyquaternium 22, such as the product Merquat 280 sold by the company Calgon;
Polyquaternium 28, such as the product Styleze CC10 sold by the company ISP;
Polyquaternium 39, such as the product Merquat Plus 3330 sold by the company Calgon;
Polyquaternium 44, such as the product Luviquat Care sold by the company BASF;
Polyquaternium 46, such as the product Luviquat Hold sold by the company BASF;
Polyquaternium 47, such as the product Merquat 2001 sold by the company Calgon.

Cationic polymers that may also be used include cationic guars, such as the product Jaguar sold by the company Rhodia.

Anionic polymers that may especially be mentioned include those comprising at least one hydrophobic chain, and in particular those derived from acrylic or methacrylic acid, for instance the acrylates/steareth-20 methacrylate copolymer sold under the name Aculyn 22 by the company Rohm & Haas (CTFA name: acrylates/steareth-30 methacrylate copolymer); (meth)acrylic acid/ethyl acrylate/oxyethylenated (25 EO) behenyl methacrylate terpolymer, as an aqueous emulsion sold under the name Aculyn 28 by the company Rohm & Haas: acrylic acid/oxyethylenated (20 EO) monocetyl itaconate copolymer, as an aqueous 30% dispersion sold under the name Structure 3001 by the company National Starch; acrylic acid/oxyethylenated (20 EO) monostearyl itaconate copolymer as an aqueous 30% dispersion sold under the name Structure 2001 by the company National Starch; acrylates/acrylate copolymer modified with polyoxyethylenated (25 EO) C12-C24 alcohols, in the form of a latex containing 30-32% copolymer, sold under the name Synthalen W2000 by the company 3V SA, and also the copolymers sold under the names Pemulen and Carbopol by the company Noveon, for instance acrylate/C10-C30-alkylacrylate copolymer such as the products Pemulen TR1, Pemulen TR2 and Carbopol 1382 (CTFA name: acrylates/C10-30 alkyl acrylate crosspolymer).

The composition may also contain polymers comprising at least one monomer containing a sulfonic group, and especially 2-acrylamido-2-methylpropane-sulfonic acid (AMPS) polymers and copolymers such as:

the crosslinked and neutralized homopolymer of 2-acrylamido-2-methylpropanesulfonic acid sold by the company Clariant under the trade name “Hostacerin AMPS” (CTFA name: ammonium polyacryldimethyltauramide);

crosslinked anionic copolymers of acrylamide or methacrylamide and of 2-acrylamido-2-methylpropane-sulfonic acid, for instance those in the form of a W/O emulsion, such as those sold under the name Sepigel 305 by the company SEPPIC (CTFA name: Polyacrylamide/C13-14 isoparaffin/Laureth-7), under the name Simulgel 600 by the company SEPPIC (CTFA name: acrylamide/sodium acryloyldimethyltaurate copolymer/isohexadecane/-Polysorbate 80),

copolymers of (meth)acrylic acid or of (meth)acrylate and of 2-acrylamido-2-methylpropane-sulfonic acid, especially those in the form of a W/O emulsion, such as those sold under the name Simulgel NS by the company SEPPIC (copolymer of acrylamido-2-methylpropanesulfonate/hydroxyethyl acrylate as a 40% inverse emulsion in Polysorbate 60 and squalane) (CTFA name: hydroxyethyl acrylate/sodium acryloyldimethyltaurate copolymer/squalane/-Polysorbate 60) or those sold under the name Simulgel EG by the company SEPPIC (copolymer of acrylic acid/acrylamido-2-methylpropanesulfonic acid in the form of the sodium salt as a 45% inverse emulsion in isohexadecane/water) (CTFA name: sodium acrylate/sodium acryloyldimethyltaurate copolymer/isohexadecane/-Polysorbate 80),

copolymers of 2-acrylamido-2-methylpropanesulfonic acid and of vinylpyrrolidone or vinylformamide, such as the products sold under the names Aristoflex AVC by the company Clariant,

hydrophobic-modified AMPS polymers, such as, especially, the copolymer of AMPS and of ethoxylated C12-C14 alkyl methacrylate (non-crosslinked copolymer obtained from Genapol LA-070 and AMPS) (CTFA name: ammonium acryloyldimethyltaurate/Laureth-7 methacrylate copolymer) sold under the name Aristoflex LNC by the company Clariant, and the copolymer of AMPS and of ethoxylated (25 EO) stearyl methacrylate) (copolymer crosslinked with trimethylolpropane triacrylate, obtained from Genapol T-250 and AMPS) (CTFA name: ammonium acryloyldimethyltaurate/Steareth-25 methacrylate crosspolymer) sold under the name Aristoflex HMS by the company Clariant.

These polymers may be in any amount (of active material) and ranging, for example, from 0.05% to 10% by weight and better still from 0.1% to 5% by weight relative to the total weight of the composition.

The compositions according to the invention may be used on any keratin material, such as the skin, the scalp, the hair, the eyelashes, the eyebrows, the nails or mucous membranes, especially as a hygiene product, for example as a product for cleansing the skin, mucous membranes and/or the hair, in particular as a cleansing and/or makeup-removing product for the skin (of the face and/or the body), as a shower product (two-in-one product), as a shampoo and hair conditioner, as a shaving product, as a rinse-off mask, as an exfoliant product (also known as a desquamating or scrubbing product), for either the face or the body or the hands, after addition of exfoliant particles.

A subject of the invention is also the cosmetic use of the composition as defined above, as a cleansing and/or makeup-removing product for the skin, as a shower product, as a shampoo or hair conditioner, as a shaving product, as a rinse-off mask or as an exfoliant product.

Another subject of the invention is a process for cleansing a keratin material, such as the skin, the scalp, the hair, the eyelashes, the eyebrows, the nails or mucous membranes, wherein a composition as defined above is applied to the keratin material and is rinsed off.

The keratin material is preferably the skin.

The compositions according to the invention may be used, for example, in the following manner:

1) when the compositions according to the invention are mild cleansing products for the face, they may be used in the following manner:

the product is worked into a lather in the hands with water

the lather is applied to the face,

the face is cleansed,

it is rinsed with water.

2) when the compositions according to the invention are makeup-removing products, they may be used as indicated above, but they may also be applied dry to the face, and then massaged until satisfactory makeup removal has been obtained, and rinsed off with water, or alternatively they may be applied using cotton-wool.

3) They may be used as a two-in-one shower product, as a shampoo and/or hair conditioner, as a rinse-off mask or as a shaving product, in the usual manner for using these products.

The examples that follow serve to illustrate the invention without, however, being limiting in nature. The amounts indicated are weight percentages, unless otherwise mentioned, and correspond to the amount of active material. The names of the compounds used are indicated as the CTFA name or as the chemical name.

### EXAMPLES

| | Example 1 | Example 2 | |
|---|---|---|---|
| | of the | of the | Comparative |
| Composition | invention | invention | example |
| Sodium lauryl glycol | 3.9 | 3.9 | 3.9 |
| carboxylate (1) | | | |
| Decylglucoside (2) | 3.25 | 3.25 | 3.25 |
| PEG-150 pentaerythrityl | 1 | 1 | 1 |
| tetrastearate (3) | | | |
| PEG-120 methylglucose | 1 | 1 | 1 |
| dioleate (4) | | | |
| PEG-14M (5) | 1 | 1 | 1 |
| N-(2-Hydroxyethyl)urea (6) | 10 | 5 | — |
| Glycerol | — | 5 | 10 |
| Preserving agents | 0.5 | 0.5 | 0.5 |
| Water | qs 100 | qs 100 | qs 100 |
| pH | 7 | 7 | 7 |
| Viscosity using a Rheomat | 1.4 Pa · s | 1.14 Pa · s | 0.67 Pa · s |
| 180 machine (spindle No. 3) | | | |
| at 200 rpm | | | |

(1) Beaulight Shaa sold by the company Sanyo
(2) Mydol 10 ®; sold by the company Kao (at 40% active material)
(3) Crothix sold by the company Croda
(4) Glucamate DOE-120 Vegetal sold by the company Amerchol
(5) Polyox WSR 205 sold by the company Amerchol
(6) Hydrovance sold by the company National Starch (as a 50% by weight aqueous solution).

Procedure: An aqueous solution of the surfactants and the preserving agents was prepared. It was heated to about 70° C. and the oxyalkylenated derivative (PEG-14M) was added. The mixture was cooled to 25° C. and the moisturizers (hydroxyethylurea and/or glycerol) were then incorporated therein. The pH was adjusted to pH 7 and the mixture was made up with water to have 100%.

The examples given above show that the cleansing compositions according to the invention have a higher viscosity than that of the comparative example that contains an amount of glycerol equivalent to that of the hydroxyethylurea in Example 1 according to the invention. The invention thus makes it possible to obtain thicker products which are thus easier to use, while at the same time having good moisturization.

### Examples 3 to 6

| | Example 3 | Example 4 | Example 5 | Example 6 |
|---|---|---|---|---|
| Sodium lauryl ether | 6.5 | — | 6.5 | — |
| sulfate | | | | |
| Cocobetaine (1) | 6.5 | 6.5 | — | — |
| Cocoglucoside (2) | — | — | 6.5 | 9.75 |
| Potassium cocoyl | — | 6.5 | — | — |
| glycinate (3) | | | | |
| Lauryl phosphate (4) | — | — | — | 3.25 |
| PEG-120 methylglucose | 1 | — | — | 1 |
| dioleate (5) | | | | |
| PEG-150 distearate | — | 2 | — | — |
| Laureth-4 (6) | — | — | 2 | — |
| PPG2 Hydroxyethyl | 1 | — | — | — |
| cocamide (7) | | | | |
| Steareth-100/PEG-136/ | — | — | — | 1 |
| HMDI copolymer (8) | | | | |
| Silica (9) | — | 5 | — | — |
| Polyquaternium-7 | 0.3 | — | — | — |
| Salicylic acid | — | 0.2 | — | — |
| KOH | — | — | — | qs pH 7 |
| N-(2-hydroxyethyl) | 10 | 2.5 | 7 | 10 |
| urea (10) | | | | |
| Citric acid | qs pH 7 | qs pH 7 | qs pH 7 | — |
| DMDM hydantoin | 0.2 | 0.2 | 0.2 | 0.2 |
| Sodium methylparaben | 0.3 | 0.3 | 0.3 | 0.3 |
| Water | qs 100 | qs 100 | qs 100 | qs 100 |

(1) Dehyton AB 30 ® sold by the company Cognis (containing 30% by weight of active material)
(2) Mydol 10 ® sold by the company Kao (at 40% active material)
(3) Amilite GCS 12 sold by the company Ajinomoto (aqueous solution containing 30% active material)
(4) MAP 20 sold by the company Kao
(5) Glucamate DOE-120 Vegetal sold by the company Amerchol
(6) Brij 30 sold by the company Uniqema
(7) Promidium CO sold by the company Uniqema
(8) Nuvis FX 1100 sold by the company Elementis
(9) Aerosil 200 sold by the company Degussa-Hüls
(10) Hydrovance sold by the company National Starch (as an aqueous solution containing 50% active material)

Products having a pleasant texture, which are easy to use and which give a good foam while at the same time being moisturizing, were obtained.

The above written description of the invention provides a manner and process of making and using it such that any person skilled in this art is enabled to make and use the same, this enablement being provided in particular for the subject matter of the appended claims, which make up a part of the original description and including a cleansing composition containing, in an aqueous medium, (1) at least one nonionic oxyalkylenated compound, (2) at least one compound of formula (I) below: 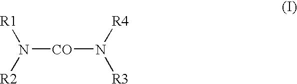
in which:
R1, R2, R3 and R4 represent, independently of each another, a hydrogen atom, a C1-C4 alkyl group or a C2-C6 hydroxyalkyl group possibly containing from 1 to 5 hydroxyl groups, in which at least one of the radicals R1 to R4 represents a hydroxyalkyl group, and also the salts, solvates and isomers thereof, and (3) at least one foaming surfactant.

As used above, the phrases “selected from the group consisting of,” “chosen from,” and the like include mixtures of the specified materials. Terms such as “contain(s)” and the like as used herein are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A composition comprising, in an aqueous medium, (1) at least one nonionic oxyalkylenated compound, (2) at least one compound of formula (I): 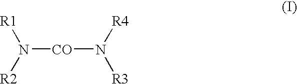
in which:
R1, R2, R3 and R4 represent, independently of each another, a hydrogen atom, a C1-C4 alkyl group or a C2-C6 hydroxyalkyl group containing from 1 to 5 hydroxyl groups, in which at least one of the radicals R1 to R4 represents a hydroxyalkyl group, and also the salts and solvates thereof, and (3) at least one foaming surfactant.

2. The composition according to claim 1, wherein it comprises at least 20% by weight of water relative to the total weight of the composition.

3. The composition according to claim 1, comprising at least one compound of formula (I) wherein R1 denotes a C2-C6 hydroxyalkyl group and R2, R3 and R4 denote, independently of each other, a hydrogen atom or a C1-C4 alkyl group.

4. The composition according to claim 1, comprising at least one compound of formula (I) wherein R1 denotes a C2-C6 hydroxyalkyl group comprising from 1 to 5 hydroxyl groups, and R2, R3 and R4 denote a hydrogen atom.

5. The composition according to claim 1, comprising at least one compound of formula (I) wherein R1 denotes a C2-C6 hydroxyalkyl group comprising 1 hydroxyl group.

6. The composition according to claim 1, comprising at least one compound of formula (I) wherein R1 denotes a C2-C4 hydroxyalkyl group comprising 1 hydroxyl group and R2, R3 and R4 denote a hydrogen atom.

7. The composition according to claim 1, comprising at least one compound chosen from N-(2-hydroxyethyl)urea; N-(2-hydroxypropyl)urea; N-(3-hydroxypropyl)urea; N-(2,3-dihydroxypropyl)urea; N-(2,3,4,5,6-pentahydroxyhexyl)urea; N-methyl-N-(1,3,4,5,6-pentahydroxy-2-hexyl)urea; N-methyl-N′-(1-hydroxy-2-methyl-2-propyl)urea; N-(1-hydroxy-2-methyl-2-propyl)urea; N-(1,3-dihydroxy-2-propyl)urea; N-[tris(hydroxymethyl)methyl]urea; N-ethyl-N′-(2-hydroxyethyl)urea; N,N-bis(2-hydroxyethyl)urea; N,N′-bis(2-hydroxyethyl)urea; N,N-bis(2-hydroxypropyl)urea; N,N′-bis(2-hydroxypropyl)urea; N,N-bis(2-hydroxyethyl)-N′-propylurea; N,N-bis(2-hydroxypropyl)-N′-(2-hydroxy-ethyl)urea; N-tert-butyl-N′-(2-hydroxyethyl)-N′-(2-hydroxypropyl)urea; N-(1,3-dihydroxy-2-propyl)-N′-(2-hydroxyethyl)urea; N,N-bis(2-hydroxyethyl)-N′,N′-dimethylurea; N,N,N′N′-tetrakis(2-hydroxyethyl)-urea; N′N′-bis(2-hydroxyethyl)-N′,N′-bis(2-hydroxy-propyl)urea; and mixtures thereof.

8. The composition according to claim 1, comprising N-(2-hydroxyethyl)urea.

9. The composition according to claim 1, wherein the salts of the compounds of formula (I) are chosen from the salts of sulfuric acid, hydrochloric acid, hydrobromic acid, hydriodic acid, phosphoric acid, boric acid, propionic acid, acetic acid, terephthalic acid, citric acid and tartaric acid.

10. The composition according to claim 1, wherein the compound of formula (I) is present in 0.1% to 50% by weight relative to the total weight of the composition.

11. The composition according to claim 1, wherein the amount of nonionic oxyalkylenated compound(s) is 1% to 10% by weight relative to the total weight of the composition.

12. The composition according to claim 1, wherein the nonionic oxyalkylenated compound is chosen from polyethylene glycols, fatty acid esters of polyethylene glycol and/or of polypropylene glycol, fatty alkyl ethers of polyethylene glycol and/or of polypropylene glycol, alkyl- or acyl-alkoxylated derivatives of polyol, glyceryl triesters of oxyalkylenated fatty acids, oxyalkylenated fatty amide derivatives and oxyethylenated urethane derivatives modified with alkyl chains, and mixtures thereof.

13. The composition according to claim 1, wherein the foaming surfactant is chosen from nonionic surfactants, anionic surfactants, and amphoteric and zwitterionic surfactants, and mixtures thereof.

14. The composition according to claim 1, comprising at least one foaming surfactant chosen from alkylpolyglucosides, betaine derivatives, alkyl glycol carboxylic acids and salts thereof, alkyl ether sulfates, alkyl phosphates, amphodiacetates, amphoacetates and alkylglycinates, and mixtures thereof.

15. The composition according to claim 1, wherein the amount of foaming surfactant(s) is 1% to 50% by weight relative to the total weight of the composition.

16. The composition according to claim 1, wherein it is a cleansing and/or makeup-removing product for the skin, a shower product, a shampoo, a hair conditioner, a shaving product, a rinse-off mask or an exfoliant product.

17. The composition according to claim 1, comprising at least one salt of a compound of formula (I) chosen from the salts of salts of sulfuric acid, hydrochloric acid, hydrobromic acid, hydriodic acid, phosphoric acid, boric acid, propionic acid, acetic acid, terephthalic acid, citric acid and tartaric acid.

18. The composition according to claim 1, comprising at least one solvate of a compound of formula (I).

19. The composition according to claim 1, comprising at least one salt of a compound of formula (I).

20. A process for cleansing a keratin material, wherein a composition according to claim 1 is applied to the keratin material, and then is rinsed off.
